(19) United States
(12) Patent Application Publication
(10) Pub. No.: US 2020/0199759 A1
(43) Pub. Date: Jun. 25, 2020

(54) SLOWLY DEGRADED ALLOY AND METHOD FOR PRODUCING THE SAME

(71) Applicant: METAL INDUSTRIES RESEARCH & DEVELOPMENT CENTRE, KAOHSIUNG (TW)

(72) Inventors: Chung-Yu HUNG, KAOHSIUNG CITY (TW); Chun-Chieh TSENG, KAOHSIUNG CITY (TW); Ying-Cheng LU, KAOHSIUNG CITY (TW); Chiu-Feng LIN, PINGTUNG COUNTY (TW); Yen-Hao CHANG, CHANGHUA COUNTY (TW)

(21) Appl. No.: 16/228,776

(22) Filed: Dec. 21, 2018

(51) Int. Cl. C23C 22/57; C23C 22/50

(52) U.S. Cl. CPC C23C 22/57; A61L 31/10; C23C 22/50

(57) ABSTRACT

The present invention relates to a slowly degraded alloy and a method for producing the same. The slowly degraded alloy comprises a degradable metal and a cladding layer. The degradable metal is completely covered with the cladding layer, such that a degradation rate of the degradable metal is decreased. Furthermore, because the cladding layer is made from polymer materials, the cladding layer does not be broken easily when a stress is applied to the slowly degraded alloy, thereby efficiently covering the degradable metal therein, and lowering the degradation rate of the degradable metal.

### BACKGROUND

### Field of Invention

The present invention relates to a bio-implanted material. More particularly, a slowly degraded alloy and a method for producing the same are provided.

### Description of Related Art

When surgical operations are applied, bio-implanted materials are often implanted to be a fixation unit or a supporter by the doctors. Although the bio-implanted materials have excellent biocompatibility, those are indwelt in the body after the surgical operations, thereby affecting and interfering following tracking and inspection. Accordingly, conventional implanted materials formed from stainless steel materials or titanium materials are replaced by bio-implanted materials formed from degradable metals.

Because the degradable metals have degrading properties, those have poor corrosion resistance. Therefore, when the degradable metals implanted in the body contact body fluids, the degradable metals are gradually corroded and dissociated to be metal ions. The metal ions can be discharged from the body by metabolism rather than remaining in the body. However, if dissociation rate of the degradable metal is too fast, a large amount of metal ions easily will poison the human tissue and harm the body. Furthermore, when the dissociation rate of the degradable metal is too fast, an evolution rate of hydrogen is increased, thereby increasing pH value of neighboring areas, such that oxygen carrying capacity of hemoglobin is lowered and abnormal reactions of the body are induced.

Generally, an oxidative protecting layer is formed on a surface of the degradable metal to isolate the degradable metal from contacting the body fluids for efficiently lowering the degradation rate of the degradable metal. However, when a stress is applied in operations or morphologies of the implanted materials are varied, the oxidative layer may be broken easily due to rigid and brittle mechanical properties, thereby exposing the degradable metal, such that the oxidative layer loses the efficacy of protecting.

In view of this, there is a need to provide a slowly degraded alloy and a method for producing the same for improving the disadvantages of the conventional slowly degraded alloy.

### SUMMARY

Therefore, an aspect of the present invention provides a slowly degraded alloy. A degradation rate of the degradable metal is reduced by covering a cladding layer on the degradable metal.

Another aspect of the present invention provides a method for producing a slowly degraded alloy. A degradable metal is immersed in a polymer solution, thereby forming a polymer layer on a surface of the degradable metal, further lowering a degradation rate of the degradable metal.

According to the aforementioned aspect, a slowly degraded alloy is provided. The slowly degraded alloy includes a degradable metal and a cladding layer. The degradable metal is completely covered by the cladding layer, and the cladding layer includes multilayers of polymer layers. A material of each polymer layers includes polysaccharide polymer.

According to one embodiment of the present invention, the cladding layer can selectively include at least one binding layers, and each of the binding layers is disposed between two adjacent layers of the polymer layers.

According to another embodiment of the present invention, the binding layers are formed from a compound having at least one carboxylic group.

According to yet another embodiment of the present invention, the compound having at least one carboxylic group includes sodium dodecyl sulfate, sodium dodecylbenzene sulfonate, glutamic acid, oxalic acid, L-amino acid and/or D-amino acid.

According to yet another embodiment of the present invention, the degradable metal includes magnesium alloy and/or Fe-based alloy.

According to yet another embodiment of the present invention, the polysaccharide polymer includes chitin, chondroitin sulfate, hyaluronic acid, glucosamine and/or starch.

According to the aforementioned aspect, a method for producing the slowly degraded alloy is provided. A degradable metal material is firstly provided and subjected to a first covering process. A degradable metal material is immersed in a polymer solution during the first covering process, so as to form a first degradable metal. The polymer solution includes polysaccharide polymer, acetic acid aqueous solution and sodium hydroxide aqueous solution. pH value of the polymer solution is less than 7. Then, a first drying step is subjected to the degradable metal immersed in the polymer solution to form a polymer layer placed on a surface of the degradable metal material, thereby obtaining the slowly degraded alloy.

According to one embodiment of the present invention, the degradable metal material is selectively immersed in an alkali solution before the first covering process is performed.

According to yet another embodiment of the present invention, the first covering process is performed at least one times.

According to yet another embodiment of the present invention, a second covering process is selectively performed between at least one of two sequential processes of the first covering processes. The first degradable metal is immersed in a binding solution to form a second degradable metal during the second covering process. The binding solution includes a compound having at least one carboxylic group and water, and a concentration of the binding solution is 0.1 wt % to 40 wt %. Then, the second degradable metal is subjected to a second drying step to form a binding layer on the polymer layer.

According to yet another embodiment of the present invention, pH value of the polymer solution is larger than or equal to 4 and less than 7.

In the slowly degraded alloy and the method for producing the same of the present invention, the degradable metal of the slowly degraded alloy is covered by specific polymer materials, so as to decrease a degradation rate of the degradable metal, thereby preventing implanted area from damages induced by too fast evolution rate of hydrogen during degrading. Moreover, when a stress is applied on the slowly degraded alloy, the tougher polymer layer is not broken easily due to strains induced by the stress, thereby efficiently preventing the degradable metal from being exposed outside.

### BRIEF DESCRIPTION OF THE DRAWINGS

The invention can be more fully understood by reading the following detailed description of the embodiment, with reference made to the accompanying drawings as follows:

FIG. 1A is a flow chart showing a method for producing a slowly degraded alloy in accordance with some embodiments of the present invention.

FIG. 1B is a schematic cross-sectional view of a slowly degraded alloy in accordance with one embodiment of the present invention.

FIG. 1C is a schematic cross-sectional view of a slowly degraded alloy in accordance with another embodiment of the present invention.

FIG. 2A is a flow chart showing a method for producing a slowly degraded alloy in accordance with some embodiments of the present invention.

FIG. 2B is a schematic cross-sectional view of a slowly degraded alloy in accordance with yet another embodiment of the present invention.

FIG. 2C is a schematic cross-sectional view of a slowly degraded alloy in accordance with yet another embodiment of the present invention.

FIG. 3 is a broken line graph of degrading experiments of slowly degraded alloys in accordance with Embodiment 1 to Embodiment 3 and Comparative Embodiment 1 of the present invention.

### DETAILED DESCRIPTION

In the following description, several specific details are presented to provide a thorough understanding of the device structures according to embodiments of the present invention. One skilled in the relevant art will recognize, however, that the embodiments of the present invention provide many applicable inventive concepts which can be practiced in various specific contents. The specific embodiments discussed hereinafter are used for explaining but not limiting the scope of the present invention.

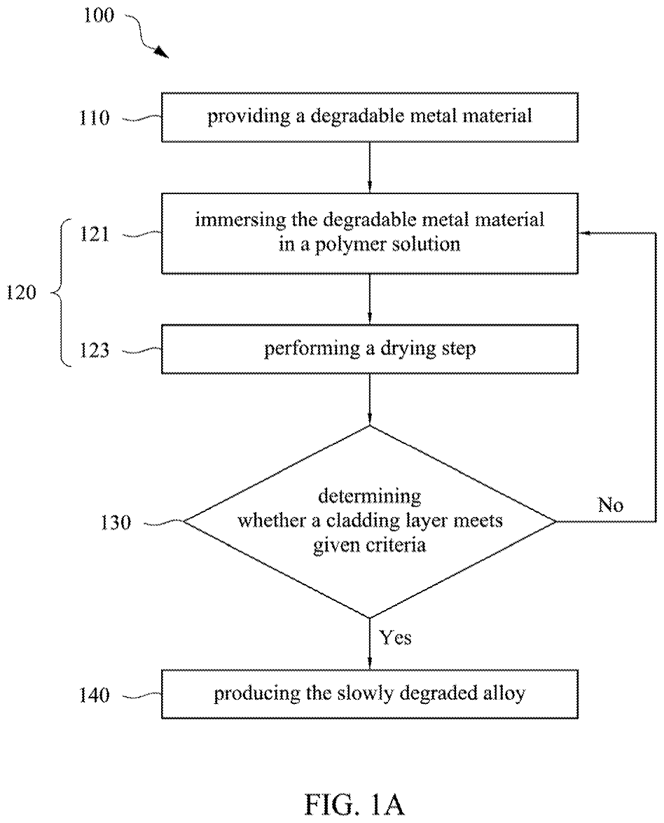

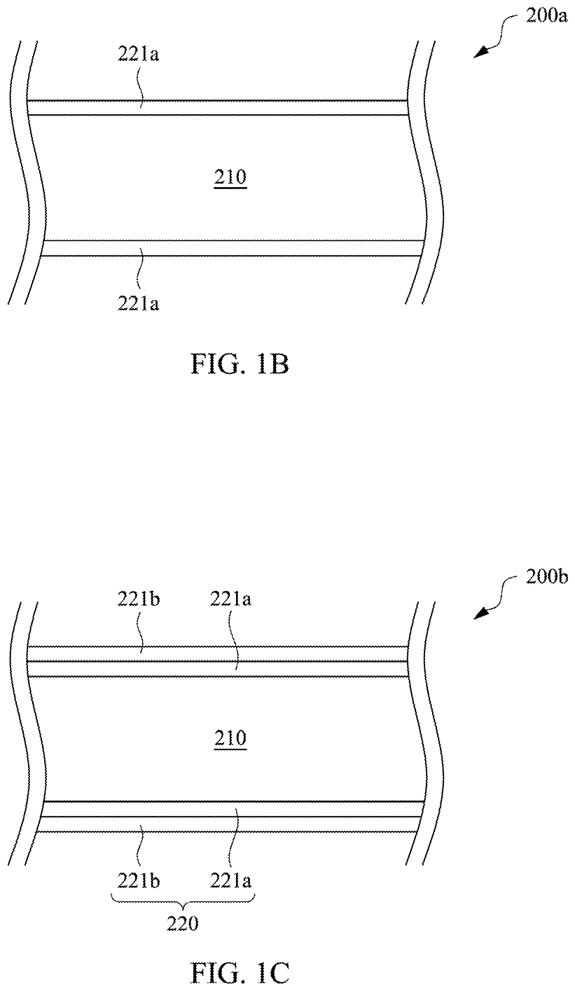

Referring to FIG. 1A and FIG. 1B. FIG. 1A is a flow chart showing a method for producing a slowly degraded alloy in accordance with some embodiments of the present invention, and FIG. 1B is a schematic cross-sectional view of a slowly degraded alloy in accordance with one embodiment of the present invention. In the method 100, a degradable metal material 210 is firstly provided, shown as operation 110. The degradable metal material 210 of the present invention is biodegradable, and metal ions produced during degradation can be completely excreted from the body by metabolism of the body. In some embodiments, the degradable metal material 210 can include but be not limited to magnesium alloy, iron based alloy, other suitable degradable metal material, or a combination thereof. In some examples, the degradable metal material 210 can include magnesium, zinc and calcium, or the degradable metal material 210 can include ferrous, zinc and calcium. For example, based on the degradable metal material 210 as 100 wt %, the degradable metal material 210 includes 84 wt % to 99.8 wt % of magnesium, 0.01 wt % to 8 wt % of zinc, and 0.01 wt % to 8 wt % of calcium.

Then, the degradable metal material 210 is subjected to a covering process 120. In the covering process 120, the degradable metal material 210 is firstly immersed in a polymer solution, shown as operation 121. The polymer solution includes polysaccharide polymer, acetic acid aqueous solution and sodium hydroxide aqueous solution, and pH value of the polymer solution is less than 7. In some embodiments, the polysaccharide polymer can include but be not limited to chitin, chondroitin sulfate, hyaluronic acid, glucosamine, starch, other suitable polysaccharide polymers, or a combination thereof.

In preparation of the polymer solution, the aforementioned polysaccharide polymer is firstly dissolved in an acetic acid aqueous solution. After the polysaccharide polymer is completely dissolved, sodium hydroxide aqueous solution is added to adjust pH value of the solution, thereby producing the polymer solution. The pH value of the polymer solution is less than 7. For example, 0.1 wt % to 10 wt % of the polysaccharide polymer is firstly added in 100 ml of acetic acid aqueous solution (a concentration of the acetic acid aqueous solution is 0.1 vol % to 30 vol %). After the polysaccharide polymer is dissolved, sodium hydroxide aqueous solution with a concentration of 1 M to 10 M is added to adjust pH value of the polymer solution. In some embodiments, pH value of the polymer solution is adjusted to be larger than or equal to 4 and less than 7.

In some embodiments, an immersion time of the degradable metal material 210 immersed in the polymer solution can be 1 minute to 60 minutes. It can be realized that there is not limited to the immersion time of the degradable metal material 210 in the polymer solution, but the surface of the degradable metal material 210 is necessary to be completely cladded by the polymer solution. In some embodiments, based on the difference of viscosities of the polymer solution, the immersion time of the degradable metal material 210 can be adjusted. In some examples, the immersion time of the degradable metal material 210 in the polymer solution can be 1 second to 1200 seconds. In some embodiments, the viscosity of the polymer solution can be 5 cp to 500 cp.

After the operation 121 is performed, a drying step is performed to the degradable metal material 210 immersed with the polymer solution, shown as operation 123. After the drying step is performed, a cladding layer 221a formed from the polymer solution is covered on a surface of the degradable metal material. In some embodiments, a drying temperature of the drying step can be 60° C. to 110° C. In some embodiments, a drying time of the drying step can be 1 hour to 2 hours.

In some embodiments, before the aforementioned covering process 120 is performed, the degradable metal material 210 can selectively be firstly immersed in an alkali solution to alkalize a surface of the degradable metal material 210. The alkali solution can include but be not limited to sodium hydroxide aqueous solution, other suitable alkali solution, or a combination thereof. In some examples, the alkali solution can be sodium hydroxide aqueous solution with a concentration of 1 M to 10 M. In those examples, an immersion time of the degradable metal material 210 in the sodium hydroxide aqueous solution can be 1 minute to 60 minutes. After the degradable metal material 210 is immersed in the alkali solution, the surface-modified degradable metal material 210 is placed and dried in an oven.

During the aforementioned covering process 120 is performed, the alkalized surface of the aforementioned degradable metal material 210 contributes to subject the polysaccharide polymer of the polymer solution to cover on a surface thereof. In addition to physical surface-cladding, the polysaccharide polymer can completely be covered on the surface of the degradable metal material 210 by intermolecular force (i.e. the force between modified groups on the alkalized surface of the surface and the polysaccharide polymer).

In some embodiments, before the degradable metal material 210 is immersed in the alkali solution, the degradable metal material 210 can selectively be subjected to a polishing process to reduce surface roughness of the degradable metal material 210. When the surface roughness of the degradable metal material 210 is reduced, a surface area of the degradable metal material 210 can be lowered, thereby reducing an area of the degradable metal material 210 where degradation-reduction reaction occurs. Therefore, when the degradable metal material 210 of the slowly degraded metal 200a is exposed outside, the degradable metal material 210 has a lower degradation rate due to the decreasing of the area where the reduction reaction occurs. In the embodiments, the polished degradable metal material 210 can be cleaned by alcohols solvent, such as ethanol or the like, and dried at 60° C. to 100° C., so as to remove residues produced during the polish process and/or the solvent or bacteria remained on the surface of the degradable metal material 210, further preventing the slowly degraded alloy from damages after implanting into the body.

After the covering process 120 is performed, the cladding layer 221a of the slowly degraded alloy 200a is determined to meet given criteria or not, shown as operation 130. In some embodiments, the given criteria can include covering thickness, numbers of the cladding layers and/or others. If the cladding layer 221a meets the given criteria, the slowly degraded alloy 200a of the present invention shown as FIG. 1B will be obtained, shown as operation 140. If the cladding layer 221a does not meet the given criteria, the covering process will be repeated, and the alloy material will be immersed in the polymer solution (e.g. the operation 121), further subjecting to the drying process (e.g. the operation 123), so as to obtain the slowly degraded alloy 200b shown as FIG. 1C.

In the slowly degraded alloy 200b, when the alloy material is immersed in the polymer solution, the dried polymer layer 221b is covered on the polymer layer 221a produced by the previous covering process 120. Accordingly, the cladding layer 220 of the slowly degraded alloy 200b can include multilayers of polymer layer 221a and polymer layer 221b. Similarly, after the polymer layer 221b is formed on the polymer layer 221a, it is necessarily to determine whether the cladding layer 220 meet the given criteria or not (i.e. the operation 130). If the alloy material does not meet the given criteria, such alloy material will be subjected to the covering process 120 again (i.e. the covering process 120 is performed for the third time). The details of the procedures are described above rather than recitation again.

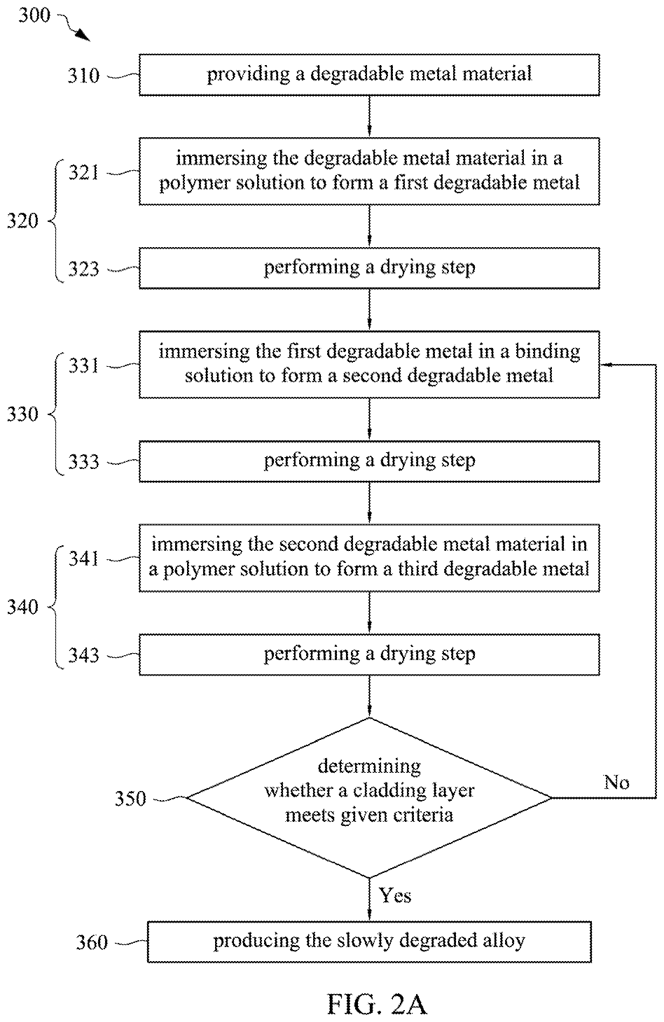

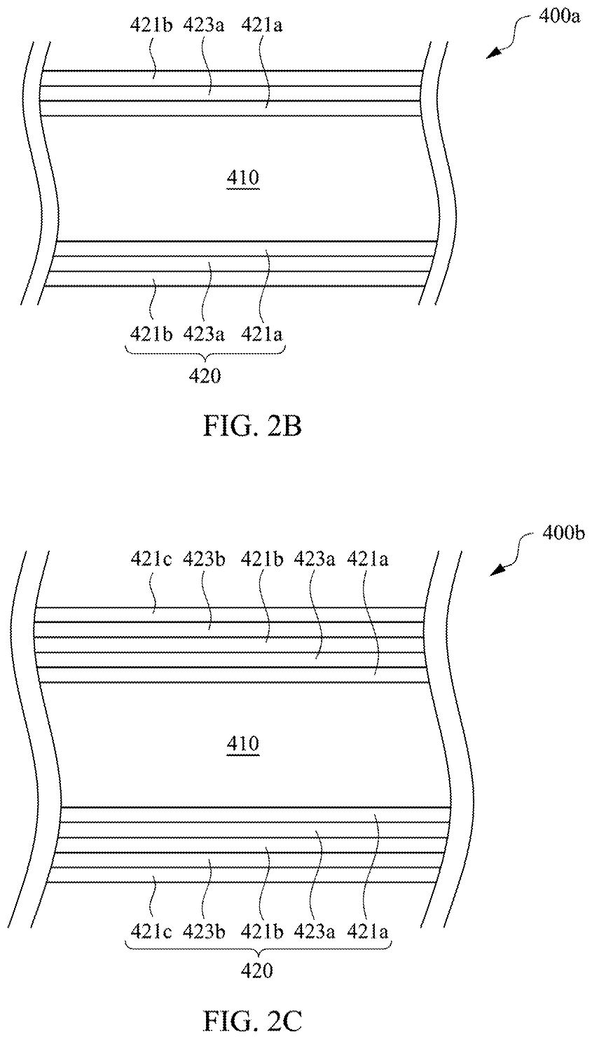

Referring to FIG. 2A and FIG. 2B. FIG. 2A is a flow chart showing a method for producing a slowly degraded alloy in accordance with some embodiments of the present invention, and FIG. 2B is a schematic cross-sectional view of a slowly degraded alloy in accordance with yet another embodiment of the present invention. A method 300 of FIG. 2A is substantially similar to the method 100 of FIG. 1A in procedures, but the difference therebetween resides in that the first degradable metal produced by the method 300 is further subjected to a second covering process 330 and a third covering process 340 after a first covering process 320 is performed.

In the second covering process 330, the first degradable metal produced by the first covering process 320 is immersed in a binding solution to form a second degradable metal, shown as operation 331. The binding solution includes a compound having at least one carboxylic group and water, and a concentration of the binding solution is 0.1 wt % to 40 wt %. In some embodiments, the compound having at least one carboxylic group can include but be not limited to sodium dodecyl sulfate, sodium dodecylbenzene sulfonate, glutamic acid, oxalic acid, L-amino acid, D-amino acid, other suitable compounds having long chain length of carbons and at least one carboxylic group, or a combination thereof.

In some embodiments, an immersion time of the first degradable metal immersed in the binding solution can be 1 minute to 60 minutes. It can be realized that there is not limited to the immersion time of the first degradable metal immersed in the binding solution, but the surface of the first degradable metal is necessary to be completely cladded by the binding solution. In other words, the binding solution is a polymer layer 421a covered on the first degradable metal (i.e. the polymer layer 421a is a polymer layer produced by the first covering process 320).

After the operation 331 is performed, a drying step is performed to the first degradable metal immersed with the binding solution, shown as operation 333. After the binding solution is dried, a binding layer 423a formed from the binding solution is covered on the polymer layer 421a. In some embodiments, a drying temperature of the drying step can be 60° C. to 110° C. In some embodiments, a drying time of the drying step can be 1 hour to 2 hours.

Then, the third covering process 340 is performed to the dried second degradable metal. In the third covering process 340, the second degradable metal produced by the second covering process 330 is immersed in the polymer solution, and a drying step is performed after the second degradable metal is immersed, shown as operation 341 and operation 343. The polymer solution used in the third covering process 340 is the same as that used in the first covering process 320. In some embodiments, the polysaccharide polymer in the polymer solution of the third covering process 340 is the same as or different from that of the first covering process 320. Moreover, the given criteria (such as immersion time, concentration of the polymer solution, drying parameters and the like) can be the same as or different from those of the second covering process 330, so as to satisfy different applications.

During the operation 343 of the third covering process 340 is performed, the polymer solution covered on a surface of the second degradable metal can be dried to form another polymer layer 421b, and the polymer layer 421b is covered on the binding layer 423a. Therefore, the binding layer 423a is placed between the polymer layer 421a and the polymer layer 421b. In other words, the cladding layer 420 of the third degradable metal includes the polymer layer 421a, binding layer 423a and the polymer layer 421b.

Then, the dried cladding layer of the third degradable metal is determined to meet given criteria or not, shown as operation 350. Similarly, the given criteria can include thickness of the cladding layer, numbers of sub-layers of the cladding layers and/or others. If the cladding layer of the third degradable metal meets the given criteria, the slowly degraded alloy 400a (shown as FIG. 2B) of the present invention will be obtained, shown as operation 360. In other words, when the cladding layer of the third degradable metal meets the given criteria, the third degradable metal is defined to be the slowly degraded alloy 400a. If the cladding layer of the third degradable metal does not meet the given criteria, the second covering process 330 and the third covering process 340 will be performed to the third degradable metal. Therefore, as shown in FIG. 2C, the polymer layer 421b of the third degradable metal is subjected to the second covering process 330 again, thereby being covered by another binding layer 423b, and then a polymer layer 421c is covered on the bonding layer 423b by the following third covering process 340.

Referring to FIG. 2A and FIG. 2C. After the polymer layer 421c is formed, the cladding layer (including the polymer layer 421a, the binding layer 423a, the polymer layer 421b, the binding layer 423b, and the polymer layer 421c) is determined again to meet the given criteria or not, shown as operation 350. If the cladding layer meets the given criteria, a slowly degraded alloy 400b of the present invention will be obtained. If the cladding layer does not meet the given criteria, the aforementioned second covering process 330 and the third covering process 340 are repeatedly performed to the alloy material having a structure shown as FIG. 2C.

In the slowly degraded alloy 400b, a surface of a degradable alloy 410 is covered by the polymer layer 421a, the binding layer 423a, the polymer layer 421b, the binding layer 423b and the polymer layer 421c in sequence. The binding layer 423a and the binding layer 423b are contributed to enhance binding properties among the polymer layer 421a, the polymer layer 421b and the polymer layer 421c, thereby increasing stability of the cladding layer 420, further improving properties of the slowly degraded alloy 400b.

It is noted that the aforementioned procedures of FIG. 2A are merely examples and are not intended to be limited. One skilled in the art can adjust the orders of the operations or omit portions of the operations based on the applications. In some embodiments, during the operation 350 of FIG. 2A is performed, if the cladding layer does not meets the given criteria, except performing the second covering process 330 to the third degradable metal again, the third degradable metal can be subjected to the first covering process 320 again, the second covering process 330 and the third covering process 340 in sequence or be repeatedly subjected to the third covering process 340. In other words, along a direction from the innermost layer (i.e. the layer mostly approaching the degradable metal material) to the outermost layer of the cladding layer, each layers of the cladding layer is not limited to that the polymer layer and the binding layer are alternative. In other embodiments, there are no binding layers among some layers of the laminated polymer layers. It is noted that the outermost layer of the cladding layer of the slowly degraded alloy is the polymer layer. If the outermost layer of the cladding layer is not the polymer layer, the outermost layer of the slowly degraded alloy is not equipped with slowly degradation effects.

In an example, the slowly degraded alloy of the present invention includes the degradable metal material and the cladding layer. Because the cladding layer is completely covered by the degradable metal material, the degradation rate of the degradable metal material can be efficiently reduced. Moreover, because the cladding layer is formed from the polymer material, the cladding layer has more toughness based on mechanical properties. Therefore, when a stress is applied on the slowly degraded alloy, the cladding layer will not be broken easily due to strains induced by the stress, thus the cladding layer can efficiently protect the degradable metal material therein. Besides, when the binding layer is disposed between two adjacent polymer layers, the binding layer can further enhance the binding properties of the polymer layers, thereby increasing the stability of the degradation rate of the slowly degraded alloy.

Several embodiments are described below to illustrate the application of the present invention. However, these embodiments are not intended to limit the scope the present invention. For those skilled in the art of the present invention, various variations and modifications can be made without departing from the spirit and scope of the present invention.

### Producing Slowly Degraded Alloy

### Embodiment 1

100 mL of an acetic acid aqueous solution with a concentration of 0.1 vol % to 30 vol % was firstly provided, and 0.1 wt % to 10 wt % of chitin was added into the acetic acid aqueous solution, thereby forming a chitin solution. A viscosity of the chitin solution was 5 cp to 500 cp. After the chitin was dissolved in the acetic acid aqueous solution, a sodium hydroxide aqueous solution of a concentration of 1 M to 10 M was added into the chitin solution to adjust pH value of the chitin solution to 4 to 7, thereby forming a polymer solution.

Moreover, a surface of a magnesium based alloy was polished and cleaned by ethanol. After the polished magnesium based alloy was dried in an oven, the magnesium based alloy was immersed in a sodium hydroxide aqueous solution with a concentration of 5 M to modify the surface. After immersing for 10 minutes, the magnesium based alloy was dried in an oven of 110° C. After drying for 1 hour, the magnesium based alloy was immersed in the aforementioned polymer solution. After immersing for 10 minutes, the magnesium based alloy was dried in an oven of 110° C. After drying for 1 hour, the magnesium based alloy with one polymer layer covered on the surface thereof was obtained.

Then, the magnesium based alloy with the polymer layer was immersed in the polymer solution again. Similarly, the magnesium based alloy was dried in the oven of 110° C. after immersing for 10 minutes. After drying for 1 hour, a slowly degraded alloy of Embodiment 1 was obtained. A cladding layer of the slowly degraded alloy of Embodiment 1 was consisted of two polymer layers. The slowly degraded alloy was evaluated according to the following evaluation method of degrading experiment, and the result thereof was listed as Table 1 rather than focusing or mentioning them in details.

### Embodiment 2 and Embodiment 3

Embodiment 2 and Embodiment 3 were practiced with the same method as in Embodiment 1, but the difference therebetween resides in that the magnesium based alloy of Embodiment 2 was immersed in the polymer solution four times and immersed in a binding solution three times, the magnesium based alloy of Embodiment 3 was immersed in the polymer solution six times and immersed in the binding solution five times, and the binding solution was a L-glutamic acid aqueous solution with a concentration of 0.1 wt % to 40 wt %.

In embodiment 2 and embodiment 3, the operations of immersing the polymer solution and the operations of immersing the binding solution were performed alternatively, such that a cladding layer of the slowly degraded alloy of embodiment 2 was consisted of four polymer layers and three binding layers alternatively placed, and the cladding layer of the slowly degraded alloy of embodiment 3 was consisted of six polymer layers and five binding layers alternatively placed. The results thereof were listed as Table 1 rather than focusing or mentioning them in details.

### Comparative Embodiment 1

The magnesium based alloy of Comparative Embodiment 1 was firstly polished and cleaned by ethanol. Then, the cleaned magnesium based alloy was placed in an oven. After the magnesium based alloy was dried, the magnesium based alloy was evaluated according to the following evaluation method of degrading experiment, and the result thereof was listed as Table 1 rather than focusing or mentioning them in details.

### Degrading Experiment

A surface area of the slowly degraded alloy of embodiment 1 to embodiment 3 and Comparative Embodiment 1 were firstly measured. Then, the slowly degraded alloy of embodiment 1 to embodiment 3 and Comparative Embodiment 1 were immersed in a same testing fluid (such as water, body fluid or the like), and evolution volume (ml/cm²) of hydrogen of the slowly degraded alloys at different immersed time were respectively measured by conventional Hydrogen Evolution Reaction.

TABLE 1

| | | | | | | | | Comparative | |
|---|---|---|---|---|---|---|---|---|---|
| | | Embodiment 1 | | Embodiment 2 | | Embodiment 3 | | Embodiment 1 | |
| | | average | standard | average | standard | average | standard | average | standard |
| | | value | deviation | value | deviation | value | deviation | value | deviation |
| Volume of | 1 day | 3.406 | 0.376 | 3.517 | 0.920 | 2.613 | 0.977 | 4.859 | 0.260 |
| hydrogen | 2 day | 7.284 | 1.210 | 6.533 | 1.008 | 5.532 | 0.482 | 11.876 | 0.915 |
| (ml/cm²) | 3 day | 11.941 | 2.313 | 10.064 | 0.899 | 9.105 | 1.687 | 18.919 | 1.556 |
| | 4 day | 16.667 | 3.657 | 13.261 | 1.106 | 12.399 | 1.765 | 24.939 | 1.650 |
| | 5 day | 23.075 | 4.724 | 18.821 | 1.120 | 17.209 | 1.644 | 30.713 | 1.963 |
| | 6 day | 28.454 | 5.062 | 24.409 | 1.622 | 21.351 | 1.188 | 36.418 | 2.232 |
| | 7 day | 34.112 | 6.396 | 29.594 | 1.794 | 26.425 | 1.183 | 42.547 | 2.631 |
| | 8 day | 39.797 | 7.877 | 34.779 | 1.966 | 31.498 | 1.314 | 48.553 | 2.370 |

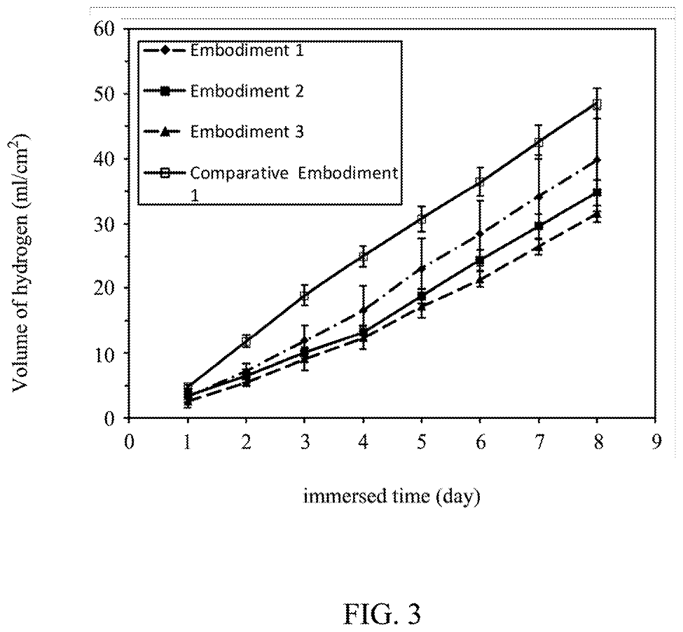

Referring to FIG. 1 and FIG. 3. FIG. 3 is a broken line graph of degrading experiments of slowly degraded alloys in accordance with Embodiment 1 to Embodiment 3 and Comparative Embodiment 1 of the present invention. X axis represents immersed time of the slowly degraded alloy, and Y axis represents evolution volume of hydrogen of the slowly degraded alloy.

According to the results of the degrading experiments of embodiment 1 to embodiment 3 and Comparative Embodiment 1, the cladding layer of the present invention can cover the degradable metal material, thereby efficiently preventing the degradable metal material from contacting the testing fluid. Moreover, when the degradable metal material was covered by at least one polymer layer, evolution volume of hydrogen of the degradable metal material was substantially decreased. Therefore, the cladding layer consisted of the polymer layers can efficiently isolate the degradable metal material from the testing fluid, thereby lowering the degradation rate of the slowly degraded alloy. As an increasing of numbers of the cladding layers, slopes of the broken lines of embodiment 1 to embodiment 3 and Comparative Embodiment 1 tended to be gentle. Therefore, the degradation rate of the slowly degraded alloy was decreased.

Besides, comparing with the cladding layer consisted of the polymer layers (i.e. Embodiment 1), a standard deviation of evolution volume of hydrogen can be substantially decreased when the cladding layer of the slowly degraded alloy further comprises binding layers (i.e. embodiment 2 and embodiment 3). Thus, binding layers contribute to enhance binding properties among the polymer layers, thereby improving stability of the slowly degraded alloy.

Accordingly, the method of producing the slowly degraded alloy of the present invention can produce the material with low degradation rate, thereby being suitable to apply in bio-implanted material. The cladding layer formed from the polymer materials can efficiently and completely cover the degradable metal material, such that the cladding layer can isolate the degradable metal material from the body fluid, thereby inhibiting the degraded reaction of the degradable metal material, further lowering the degradation rate. Moreover, the binding layer can form between adjacent polymer layers in the cladding layer by the operation of immersing in the binding solution, thereby increasing binding properties of the adjacent polymer layers, and further enhancing stability of the slowly degraded alloy. Because the cladding layer is formed from the polymer materials, the cladding layer is tough. Therefore, when a stress is applied on the slowly degraded alloy, the cladding layer is not broken easily due to strains induced by the stress. Thus, the cladding layer can efficiently protect the degradable metal material.

As is understood by a person skilled in the art, the foregoing embodiments of the present invention are illustrative of the present invention rather than limiting the scope of the present invention. It is intended to cover various modifications and similar arrangements included within the spirit and scope of the appended claims, the scope of which should be accorded with the broadest interpretation so as to encompass all such modifications and similar structures.

What is claimed is:

1. A slowly degraded alloy, comprising:
a degradable metal; and
a cladding layer, wherein the degradable metal is completely covered by the cladding layer, the cladding layer includes a plurality of polymer layers, and a material of each of the polymer layers includes polysaccharide polymer.

2. The slowly degraded alloy of claim 1, wherein the cladding layer further comprises:
at least one binding layers, wherein each of the binding layers is disposed between two adjacent layers of the polymer layers.

3. The slowly degraded alloy of claim 2, wherein the at least one binding layers are formed from a compound having at least one carboxylic group.

4. The slowly degraded alloy of claim 3, wherein the compound having at least one carboxylic group includes sodium dodecyl sulfate, sodium dodecylbenzene sulfonate, glutamic acid, oxalic acid, L-amino acid and/or D-amino acid.

5. The slowly degraded alloy of claim 1, wherein the degradable metal includes magnesium alloy and/or Fe-based alloy.

6. The slowly degraded alloy of claim 1, wherein the polysaccharide polymer includes chitin, chondroitin sulfate, hyaluronic acid, glucosamine and/or starch.

7. A method for producing a slowly degraded alloy, comprising:
providing a degradable metal material; and
performing at least one first covering process, wherein the first covering process includes:
immersing the degradable metal material in a polymer solution, thereby forming a first degradable metal, wherein the polymer solution includes polysaccharide polymer, acetic acid aqueous solution and sodium hydroxide aqueous solution, and pH value of the polymer solution is less than 7; and
performing a first drying step to the first degradable metal to form a polymer layer on a surface of the degradable metal material, thereby producing the slowly degraded alloy.

8. The method for producing the slowly degraded alloy of claim 7, after the first covering process is performed, further comprising:
immersing the degradable metal material in an alkali solution.

9. The method for producing the slowly degraded alloy of claim 8, wherein the first covering process is performed a plurality of times, a second covering process is further performed therebetween the first covering process, and the second covering process comprises:
immersing the first degradable metal in a binding solution to form a second degradable metal, wherein the binding solution includes a compound having at least one carboxylic group and water, and a concentration of the binding solution is 0.1 wt % to 40 wt %; and
performing a second drying step to the second degradable metal to form a binding layer on the polymer layer.

10. The method for producing the slowly degraded alloy of claim 7, wherein pH value of the polymer solution is larger than or equal to 4 and less than 7.
